(19) United States
(12) Patent Application Publication
(10) Pub. No.: US 2024/0027459 A1
(43) Pub. Date: Jan. 25, 2024

(54) METHOD OF DETERMINING LONG-TERM SURVIVAL OF CANCER PATIENTS WITH OVARIAN CANCER

(71) Applicant: Katholieke Universiteit Leuven, Leuven (BE)

(72) Inventors: Patrizia AGOSTINIS, Kessel-Lo (BE); An COOSEMANS, Heverlee (BE); Abhishek GARG, Herent (BE); Jenny SPROOTEN, Heverlee (BE)

(21) Appl. No.: 18/042,367

(22) PCT Filed: Aug. 19, 2021
(86) PCT No.: PCT/EP2021/073091

§ 371 Date: Feb. 21, 2023

(30) Foreign Application Priority Data
Aug. 21, 2020 (EP) 20192170.7

(51) Int. Cl. G01N 33/574; G01N 33/50

(52) U.S. Cl. CPC G01N 33/57488; G01N 33/5091; G01N 2500/10

(57) ABSTRACT

The invention relates to in vitro methods for predicting tumour progression or response to anticancer therapy of a cancer patient the method comprising the steps of: 1) providing engineered mammalian immune cells comprising: —a NFkB signalling reporter construct and an IFN signalling reporter construct, 2) contacting the engineered cells with a blood sample of a cancer patient, allowing induction of the NFkB and/or IFN signalling pathways of said engineered cells by the blood sample, 3) determining the expression and/or activity levels of the first and second reporter protein, 4) comparing the expression levels and/or activity of the first and second reporter protein determined in step 3) with the expression and/or activity levels of the first and second reporter protein in a reference sample of a healthy individual, 5) based on the comparison predicting tumour progression, or response to anticancer therapy of the cancer patient.

### FIELD OF INVENTION

This invention relates to the field of immuno-oncology serum-based biomarkers to predict long-term tumour progression in general (prognostic) or in response to various classes of anticancer therapy (predictive).

### BACKGROUND OF THE INVENTION

Tumour stage, residual disease after initial surgery, histological type and tumour grade are the most important clinico-pathological biomarkers related to the clinical outcome of cancer patients [Gadducci et al. (2009) Crit. Rev. Oncol. Hematol. 69, 12-27; Vošmik et al. (2018) Pathol Oncol Res. 24, 373-383] Beyond these, specific biomarkers are often utilised either for long-term estimation of cancer patient survival (i.e. prognostic biomarkers) or to guide the application of, or monitor the responses to, different anticancer therapies (i.e. predictive biomarkers) [Nalejska et al. (2014) Mol Diagn Ther. 18, 273-284; Califf (2018) Exp. Biol. Med. 243, 213-221]. The use of such prognostic or predictive biomarkers gained a huge momentum recently with the emergence of cancer immunotherapy [Yuan et al. (2016) J. Immunother. Cancer 4, 3; Shindo et al. (2019) Cancers (Basel) 11, 1223]. This was because several highly efficacious immunotherapies were found to work better in patients possessing a combination of broad tumour-level biomarkers like high tumour mutational/neoantigen burden (TMB), high PD-L1 levels and high tumour-infiltrating lymphocytes (TILs) [Cristescu et al. (2018) Science. 362 eaar3593; Fumet et al. (2020) Eur. J. Cancer 131, 40-50]. In general, broad biomarkers (like the ones mentioned above) have been found to predict patient responses to immunotherapy better than very specific biomarkers (e.g. TCR or BCR clonality) [Topalian et al. (2020) Science 367 eaax0182; Pardoll (2012) Nat. Rev. Cancer. 12, 252-264; Havel et al. (2019) Nat. Rev. Cancer 19, 133-150]. However, a major drawback of these tumour-level biomarkers is: (1) their detection is highly invasive thereby making repeated detections problematic; and (2) tumour-material may not be always available at every phase of patient monitoring and follow-up to inform clinicians thereby hampering long-term usage of these biomarkers for immuno-oncology. To overcome these issues, the field of immuno-oncology is starting to intensively consider less invasive sources of good biomarkers like those that can be detected in patient serum [Ferreira et al. (2019) Curr Drug Targets 20, 81-86; Mehnert et al. (2017) Clin. Cancer Res. 23, 4970-4979]. In the last years, several investigations have assessed different biological variables in sera from cancer patients in order to detect biomarkers able to reflect either the response to chemotherapy, targeted therapy or immunotherapy and/or survival. Predictive, monitoring or prognostic relevance of various serum-associated cytokines, chemokines, metabolites and specific immune cell phenotypes has been assessed and, in some case, validated for clinical use in immuno-oncology context e.g. serum levels of IL6 or CRP [Chalabi et al. (2020) Nat. Med. 26, 566-576; Versluis et al. (2020) Nat. Med. 26, 475-484]. However, this progress is far too limited, as several of these biomarkers are not always reliable and their contextual activities are not completely elucidated. For example, the interaction between tumour and immune system/stromal cells, the production of cytokines/chemokines by the tumour itself and the peripheral immune interactions can result in different local and systemic levels of cytokines/chemokines in cancer patients. Several studies have shown that most cytokines/chemokines are not independent prognostic biomarkers, and individual cytokines are not specific/reliable enough for screening purposes. And although several new multiplex techniques like, the cytokine bead array (CBA), Luminex or MSD platform, have been developed for simultaneous analysis of multiple cytokines/chemokines in serum samples; yet these technical advancements have not entirely overcome the biological and clinical limitations of immunological serum biomarkers.

All these multiplexed serum immunological biomarker assays tend to have a largely quantitative approach toward biomarker identification. This creates a big hurdle in estimating the patient serum's “phenome” (a sum of all phenotypic traits) since correct phenome-mapping requires both quantitative as well as qualitative (i.e. functional or effector) estimation of serum factors. The outright caveat of quantitative biomarker-estimation approach for immunological factors is that, quantity for a given cytokine/chemokine may not be linearly associated with their actual biological functions. Cytokines/chemokines linearly exert their specific functions only up to a threshold of concentration (which is specific for each cytokine/factor, its biological context as well as diseased tissue), beyond which their effects tend to exert a self-regulating function thereby causing functional “plateau” or even suppression. This situation is further complicated by the fact that, in full serum derived from cancer patients, immuno-stimulatory, immuno-suppressive and, immuno-homeostatic cytokines, haematopoietic factors or chemokines are simultaneously present. Thus, factors with contradictory or complementary immunological functions co-exist in the serum at the same time. This situation is further complicated by post-translational modifications of cytokines/chemokines that can drastically alter their immunological functions. Finally, there are several known or unknown biomolecular entities that are not covered (or cannot be reliably covered) by routinely applicable multiplexed technologies. Accordingly, multi-parametric detection of all (or some of) these factors via multiplexed technologies, frequently ends up creating a problematic situation with regard to serum phenome-mapping i.e., detection of contradictory (functional or prognostic/response/predictive) trends for these cytokines, chemokines or factors, which makes it hard to draw concise immunological conclusions about the therapy-responsiveness and immunological status of the patient. Although efforts are being made to overcome these limitations via application of advanced computational or bioinformatics approaches, yet these tend to fail in predicting non-linear characteristics of immunological pathways e.g. oscillations, chaos and concentrations-dependent non-linearity. Thus, deciphering the functionally “integrated immunological activity” of these cytokines/chemokines/factors remains unknown based only on the quantitative measurement of these individual proteins (as is currently the standard practice in the field everywhere). This in turn makes it almost impossible to reliably map a patients' “Immunological Serum Biomarkers” (ISB)-based phenotype. In the field of serum biomarkers in oncology currently, actionable analysis of functional immunological status of cancer patients' sera is entirely absent on the level of soluble factors.

### SUMMARY OF THE INVENTION

The invention relates to functional immunological biomarker assay to reliably estimate the ISB-phenotype in cancer patients. Specifically, this biomarker modality (i.e. serum-based functional immunological assay or sFIS assay) consists of two of the most major serum-relevant immunological pathways (i.e. Nuclear factor kappa-light-chain-enhancer of activated B cells or NFkB and, interferon or IFNs responses) detected on the level of genetic reporter monocytes of human origin, exposed to the serum derived from cancer patients. This sFIS assay is particularly efficient at simultaneously estimating differential long-term survival of cancer patients. It simultaneously integrates both negative and positive prognostic/predictive biomarker modalities in the same assay.

The present invention is summarised in the following statements.

1. An in vitro method for determining the health status of a cancer patient, the method comprising the steps of:
1) Providing engineered mammalian cells comprising:
   a reporter gene construct for NFkB response signalling, and/or
   a reporter gene construct system for IFN response signalling,
2) Contacting the engineered cells with a body sample, typically blood sample, of a cancer patient, allowing induction of the NFkB and/or IFN signalling pathways of said engineered cells by the blood sample;
3) Determining the expression levels of the reporter gene and/or activity of the expressed reporter gene for NFkB signalling, and/or determining expression levels of the reporter gene and/or activity of the reporter gene for IFN signalling,
4) Comparing the expression levels and/or activity determined in step 3) with the expression and/or activity levels of reference sample of a healthy individual,
5) Based on this comparison determining the health status of the cancer patient. Herein, engineered cells are transiently or stably transfected with a vector carrying the reporter gene construct.

Herein the blood sample is typically a processed blood sample such as plasma, or preferably serum. Alternatively, the processed blood sample is a purified protein or lipid fraction obtained from the blood sample. Or a physiologically generated blood-filtered fluid e.g., ascites fluid, which is blood filtered fluid that builds-up in the patient's peritoneum.

The method can be performed prior, after or during therapy as well as at first or recurrence diagnosis stage.

2. The method according to statement 1, wherein the health status predicts the life expectancy of the cancer patient after cancer therapy.

3. The method according to statement 1 or 2, wherein the reporter gene construct for NFkB signalling, and the reporter gene construct system for IFN signalling, are in the same cell.

4. The method according to statement 1 or 2, wherein the reporter gene construct for NFkB signalling, and the reporter gene construct system for IFN signalling, are in different cells.

5. The method according to any one of statements 1 to 4, wherein the cells are primate cells, preferably human cells.

For example, rodent cells can be used when they express human immune receptors or sensors upstream of the reporter constructs.

6. The method according to any one of statements 1 to 5, wherein the cells in step 1) are monocytes.

Other suitable cells are for example macrophages, dendritic cells, granulocytes, T cells, B cells, NK cells, lymphocytes, myeloid cells, cancer cells, malignant cells, adenoma cells, carcinoma cells, neoplastic cells, virally or chemically transformed cells, epithelial cells, fibroblasts and embryonic cells.

. The method according to any one of statements 1 to 6,
wherein a ≥1.5 fold-change of expression level of the reporter gene and/or activity of the expressed reporter gene for NFKb signalling compared to the reference value is a negative indicator of the health status of the cancer patient (the cancer patient is likely to show a shortened overall survival).

In an alternative method a ≥75th percentile of overall data point distribution in a patient screening data set is used to make the assessment.

8. The method according to any one of statements 1 to 6, wherein a ≥1.3 fold-change of expression level of the reporter gene and/or activity of the expressed reporter gene for NFKb signalling compared to the reference value is a positive indicator of the health status of the cancer patient. (the cancer patient is likely to show a prolonged overall survival).

In an alternative method a ≥75th percentile of overall data point distribution in a patient screening data set is used to make the assessment.

The method according to any one of statements 1 to 6, where the reporter gene is a luciferase, a fluorescent or bioluminescent protein or an alkaline phosphatase

9. The method according to any one of statements 1 to 8, wherein the expressed reporter gene is a secreted protein

10. The method according to any one of statements 1 to 9, wherein the IFN reporter protein is under the control of an interferon-induced transcription factors-responsive promoter linked to one or more (e.g. 5) copies of IFN-stimulated response element (ISRE) sequences.

Interferon-induced transcription factors are for example members of the STAT family proteins and members of the IRF family.

11. The method according to any one of statements 1 to 9, wherein the NFKB reporter protein is under the control of a NFkB signalling complex-responsive promoter linked to multiple (e.g. three) copies of c-REL binding site and multiple (e.g. five) copies of the NFKB consensus transcriptional response element.

NFkB signalling complex-responsive protein are for example NFkB p50/p65 or members of the REL family.

12. The method according to any one of statements 1 to 11, wherein the patient has ovarian cancer, malignant ovarian cancer or high-grade serous ovarian carcinoma.

14. The method according to any one of statements 1 to 12, wherein the patient underwent an anticancer therapy selected from the group consisting of chemotherapy, radiotherapy, small-molecule inhibitors, targeted therapy, palliative therapy, alternative therapies and immunotherapy.

15. Use of engineered human cells comprising a reporter gene construct for NFkB signalling, and/or a reporter gene construct system for IFN signalling, in determining the health status of a cancer patient.

16. An in vitro method for predicting tumour progression or response to anticancer therapy of a cancer patient, wherein the cancer is selected from the group consisting of ovarian cancer, cervical squamous cell carcinoma and endocervical adenocarcinoma (CESC), head and neck squamous cell carcinoma (HNSC) and liver hepatocellular carcinoma (LHC), the method comprising the steps of:
1) providing engineered mammalian immune cells comprising:
   a NFkB signalling reporter construct comprising an NFkB signalling complex-responsive promoter sequence linked to multiple copies of c-REL binding site and multiple copies of the NFKB consensus transcriptional response element, which binds an NFkB transcription factor complex, fused to a gene sequence encoding a first reporter protein, and
   an IFN signalling reporter construct comprising an interferon-induced transcription factors-responsive promoter sequence linked to one or more copies of IFN-stimulated response element (ISRE) sequences, which binds IFN response transcription factors, fused to a gene sequence coding a second reporter protein,
2) contacting the engineered cells with a blood sample of a cancer patient, allowing induction of the NFkB and/or IFN signalling pathways of said engineered cells by the blood sample,
3) determining the expression and/or activity levels of the first and second reporter protein,
4) comparing the expression levels and/or activity of the first and second reporter protein determined in step 3) with the expression and/or activity levels of the first and second reporter protein in a reference sample of a healthy individual,
5) based on the comparison predicting tumour progression, or response to anticancer therapy of the cancer patient,
wherein an increase of activity or expression level of the first reporter protein for NFkB signalling compared to the reference value is a negative indicator of tumour progression, or response to anticancer therapy, and
wherein an increase of activity or expression level of the second reporter gene for IFN signalling compared to the reference value is a positive indicator of the tumour progression, or response to anticancer therapy.

17. The method according to statement 16, wherein the response to anticancer therapy predicts the life expectancy of the cancer patient after cancer therapy.

18. The method according to statement 16, wherein the prediction of tumour progression predicts the medium-to-long term survival of a cancer patient.

19. The method according to any one of statements 16 to 18, wherein the immune cells are human cells.

20. The method according to any one of statements 16 to 19, wherein the immune cells in step 1) are monocytes.

21. The method according to any one of statements 16 to 20, wherein a 1.5 fold-change of expression and/or activity of the first reporter protein for NFkB signalling, compared to the reference value, is a negative indicator of tumour progression or response to anticancer therapy.

22. The method according to any one of statements 16 to 20, wherein a 1.3 fold-change of expression level and/or activity of the second reporter protein for IFN signalling, compared to the reference value, is a positive indicator of the tumour progression or response to anticancer therapy.

23. The method according to any one of statements 16 to 22, where the reporter protein is selected from the group consisting a luciferase, a fluorescent or bioluminescent protein and an alkaline phosphatase.

24. The method according to any one of statements 16 to 23, wherein the expressed reporter gene is a secreted protein.

25. The method according to an one of statements 16 to 24, wherein the ovarian cancer is malignant ovarian cancer or high-grade serous ovarian carcinoma.

26. The method according to any one of statements 16 to 25, wherein the patient underwent an anticancer therapy selected from the group consisting of chemotherapy, radiotherapy, small-molecule inhibitors, targeted therapy, palliative therapy, alternative therapies and immunotherapy.

27. Use of an engineered mammalian cells comprising a NFkB reporter gene construct, and an IFN reporter gene construct,
a NFkB signalling reporter construct comprising an NFkB signalling complex-responsive promoter sequence linked to multiple copies of c-REL binding site and multiple copies of the NFKB consensus transcriptional response element binding an NFkB transcription factor complex, fused to a gene sequence encoding a first reporter protein, and
an IFN signalling reporter construct comprising an interferon-induced transcription factors-responsive promoter sequence linked to one or more copies of IFN-stimulated response element (ISRE) sequences, which binds IFN response transcription factors, fused to a gene sequence coding a second reporter protein,

in predicting tumour progression or response to anticancer therapy of a cancer patient, wherein the cancer is selected from the group consisting of ovarian cancer, cervical squamous cell carcinoma and endocervical adenocarcinoma (CESC), head and neck squamous cell carcinoma (HNSC) and liver hepatocellular carcinoma (LHC).

Control measurements are performed using serum from a healthy person (i.e. without cancer), typically using a pool of serum of several persons. The pooling of control sera avoids bias by an undiagnosed cancer, by a condition other than cancer which would effect NFkB or IFN signalling, and averages for age and sex, and other variations in the population.

The expression of reporter genes is determined directly by detecting the presence of mRNA (rtPCR) of or the expressed protein via e.g. the fluorescence of a Green Fluorescent Protein, or by an antibody assays,

Alternatively, when the reporter gene encodes an enzyme, the activity of the enzyme is a used as measure of the expressed protein.

In certain embodiments the expressed protein is an secreted protein, such that the expression or activity is determined in the medium of the cell culture.

In other embodiments, the expressed protein is an intracellular protein, and the product of the enzymatic reaction is secreted.

In other embodiments, the expressed protein is an intracellular protein and the protein and/or reaction products of an enzymatic reaction are detected by microscopic methods, or by cell sorting or, are determined in the cell lysate.

A non-limiting list of reporter genes includes fluorescent proteins, enzymes capable of generating colorimetric products, or enzymes capable of generating fluorescent products, such as luciferase or alkaline phosphatase. When both reporter genes are in the same cell, reporter genes are selected such that they can be individually detected.

The methods of the present invention are suitable on various types of cancer such as ovarian cancer, lung cancer, cervical cancer, leukaemia, lymphoma, melanoma, sarcoma, bladder cancer, renal cell cancer, breast cancer, colorectal cancer, brain cancer, urothelial cancer, kidney cancer, cancers of epithelial origin and cancers with circulating metastatic cancer cells.

The methods of the present invention allow to evaluate the health status of a patient undergoing or having completed a therapy such as chemotherapy, radiotherapy, immunotherapy, targeted therapy, surgery, palliative therapy, herbal therapy, therapy with anticancer vaccines, oncolytic viruses, cytokines/chemokines, recombinant proteins or physico-chemical therapeutics.

### DETAILED DESCRIPTION OF THE INVENTION

### Description of the Figures

Figure Legends:

FIG. 1. Violin plots of IFNs signalling signature (A-B) or NFkB signalling signature (C-D) in pre-chemotherapy (platin/taxane/gemcitabine/topotecan) treated Stage III OV-tumour tissue available from (serous) OV-patients that underwent optimal debulking. These were subdivided as responders (n=77) or non-responders (n=24) based on pathological responses (A-C) as well as responders (n=99) or non-responders (n=37) based on relapse free survival (RFS) (B-D) at 1 year follow-up as per availability (Mann-Whitney test; two-tailed; *p<0.05). The data in D was utilized for ROC curve analyses (AUC: area under curve; *p<0.05) (E). Data for A-E were accessed from a large integrated database that pooled OV-patient datasets form GEO and TCGA repositories with available treatment and patient response information [Fekete et al. (2020) Gynecol. Oncol. 156, 654-661]. (F) Violin plot of NFkB signalling signature in ascites cellular samples obtained from pre/on-immunotherapy (i.e. infliximab, an anti-TNF antibody) treated OV-patients [Kulbe et al. (2012) Cancer Res. 72, 66-75]. These were subdivided as responders (n=4) or non-responders (n=5) (Mann-Whitney test; two-tailed; *p<0.05).

FIG. 2. Human THP1 monocytes binary reporter cells were exposed to indicated concentrations of the natural agonists for TLR4 (LPS), RIG-I (5′ppp-dsRNA+LysoVec) or STING (2′3′-cGAMP) and THP1 monocytes' reporting of NFkB (A) or IFNs (B) responses was captured at 48 h post-treatment (n=3; One-sample t-test; *p<0.05).

FIG. 3. Human THP1 monocytes binary reporter cells were exposed to indicated concentrations of various human recombinant cytokines (A-B) or immune-checkpoint molecules (C-D) and THP1 monocytes' reporting of NFkB (A-C) or IFNs (B-D) responses was captured at 48 h post-treatment (n=3).

FIG. 4. (A) Quantitatively as well as qualitatively suitable serum samples from the UZL-CSI cohort were profiled for the serum-induced NFkB or IFNs response via the sFIS assay (n=96, Wilcoxon matched-pairs signed rank test; *p<0.05). B depicts the paired sample-representation for the simultaneous NFkB and IFNs response within the sFIS assay such that “§” depicts serum samples wherein the IFNs response values exceeded the NFkB response values.

FIG. 5. Cubic spline analyses of sFIS assay readouts (NFkB or IFNs responses) (A-B) or CA125 concentration (C-D) profiled from the serum of the UZL-CSI ovarian cancer cohort and distributed as per PFS (A-C) or OS values (B-D). In cases where there were multiple serum specimens per patient, median across specimens were considered to derive a singular value for different analytes per patient.

FIG. 6. Violin plots of sFIS assay readouts i.e. NFkB responses or IFNs responses and CA125 concentration (A-B) (median levels per patient) profiled from the serum of the UZL-CSI ovarian cancer cohort. These were subdivided as longer-PFS (A) or longer-OS (B) and shorter-PFS (A) or shorter-OS (B) based on a cut-off of 2 years of PFS or 3 years of OS (CA125, longer-PFS n=5 vs. shorter-PFS n=26; NFkB/IFNs Resp., longer-PFS n=5 vs. shorter-PFS n=27; Mann-Whitney test; two-tailed; *p<0.05). The data in A-B were utilized for ROC curve analyses (AUC: area under curve; *p<0.05) (C-D).

FIG. 7. Kaplan-Meier (KM) plots of CA125 concentration or sFIS assay readouts i.e. NFkB responses or IFNs responses (A-B) (median levels per patient) profiled from the serum of the UZL-CSI ovarian cancer cohort. These were analysed for PFS (A) or OS (B) on the basis of subdivision as HIGH or LOW (A-B) levels based on a cut-off at the 75th percentile of overall value distributions for CA125 or sFIS assay readouts (CA125 vs. PFS/OS, HIGH n=8 vs. LOW n=23; NFkB/IFNs Resp. vs. PFS/OS, HIGH n=8 vs. LOW n=24). The plots depict the Hazard Ratio (HR)±95% confidence interval (CI) (Log-rank Mantel-Cox test; *p<0.05).

FIG. 8. Representation of the levels of NFkB-target proteins, IFNs-related proteins profiled from the serum of the UZL-CSI ovarian cancer dataset and subdivided on the basis of different before-treatment or on/post-treatment subgroups from which the 98 serum samples across the 32 ovarian cancer patients are derived, within the UZL-CSI cohort.

FIG. 9. Expression plots of CA125 concentration and sFIS assay readouts i.e. NFkB responses or IFNs responses profiled from the serum of the UZL-CSI ovarian cancer cohort (A), subdivided based on different pathological responses i.e. complete (CR) or partial (PR) responses and stable (SD) or progressive (PD) disease accessed at serum-collection time-points (CA125, CR n=12, PR n=14, SD n=10, PD n=50; NFkB/IFNs Resp., CR n=13, PR n=14, SD n=10, PD n=49). (B) The above data was also utilized to create violin plots, where responder patients (CR) vs. non-responder patients (PD) are compared (CA125, CR n=12 vs. PD n=50; NFkB/IFNs Resp., CR n=13 vs. PD n=49; Mann-Whitney test; two-tailed; *p<0.05). The data in B were utilized for ROC curve analyses (AUC: area under curve; *p<0.05) (C).

FIG. 10. (A) Violin plot of the OV-patients' OS or PFS distributions for patients treated with paclitaxel+carboplatin based combinatorial regimens (COMBOs) (n=14) or Bevacizumab alone (n=11) in the UZL-CSI cohort. (B) A Spearman correlation analyses between OV-patients' PFS or OS (within the treatment subgroups of paclitaxel+carboplatin COMBOs or Bevacizumab) with concentration trends of the indicated and sFIS assay readouts (NFkB or IFNs responses) and CA125 profiled from the serum of the UZL-CSI ovarian cancer dataset. In cases where there were multiple serum specimens per patient, median across specimens was considered to derive a singular value for different analytes per patient.

FIG. 11. Correlation plots of Neutrophil-to-Lymphocytes ratio (NLR) scores profiled from whole blood of the UZL-CSI ovarian cancer cohort vs. serum CA125-levels (n=63) (A), or sFIS assay readouts of serum-induced NFkB Resp. (n=64) (B) or IFNs Resp. (n=64) (C); The plots depict Pearson correlation±95% confidence interval (CI); p<0.05.μ

FIG. 12. Visualization of the HR±95% confidence interval (CI) for the impact of expression of NFkB signalling gene signature or IFN signalling gene signature, for cancer datasets wherein the signature expression cut-off for binary (high vs. low expression) patient stratification was based on best performing threshold principle for overall survival (OS) of indicated TCGA cancer patients (LIHC; n=371, PAAD; n=177, LUSC; n=501, LUAD; n=513, HNSC; n=500, CESC; n=304, BLCA; n=405, UCEC; n=543, OV; n=374, SARC; n=259, BRCA; n=1090, KIRC; n=530) (Mantel-cox test; *p<0.05).

FIG. 13. The sFIS assay-based prediction of chemo-immunotherapy regime's design and in vivo testing in murine metastatic ovarian cancer model. (A) Overview of the tumour inoculation and therapeutic treatment schedules for the mice experiments. (C) si-IFN and si-NFkB response of J774 dual-reporter cell lines exposed to mouse serum obtained from day 49 as a ratio to day 13 (Control; n=8, anti-TNF Ab; n=8, PARPi; n=7; PARPi+anti-TNF Ab; n=8, PTX+CBP; n=7, PTX+CBP+anti-TNF Ab; n=6). (C,D) Kaplan-Meier plots of overall survival (C), or survival while considering the first drainage of ascitic fluid (D) of metastatic ID8 tumour bearing mice treated with different therapy regimes (Control; n=8, anti-TNF Ab; n=8, PARPi; n=8; PARPi+anti-TNF Ab; n=8, PTX+CBP; n=7, PTX+CBP+anti-TNF Ab; n=7)(Log-rank Mantel-Cox test; *p<0.05; **p<0.01).

The present invention shows that the major ISB-based phenotype-defining pathways largely come down to innate inflammatory (NFkB inflammatory response) or effector innate/adaptive immune pathways (IFNα-β/IFNγ response), mainly operating via either NF-kB response modules (NFkB or REL) or an IFN response module (ISRE-binding TFs, IRF, IRF1/8). Thus, the “core” ISB-based phenotype in cancer patients seems to consist of various serum-associated cytokines or immunological factors largely engaging the NFkB signalling or IFNs signalling immune-pathways.

Cancer serum biomarkers can in fact proficiently induce NFkB and/or IFN signalling, however there is no minimal set of genes that can be used as a diagnostic modality for a broad range of cancer types. This problem is addressed in the present invention, by using the intracellular downstream signalling, which represents the overall response of stimulation/inhibition as a prognostic/predictive tool rather than using the individual signalling proteins.

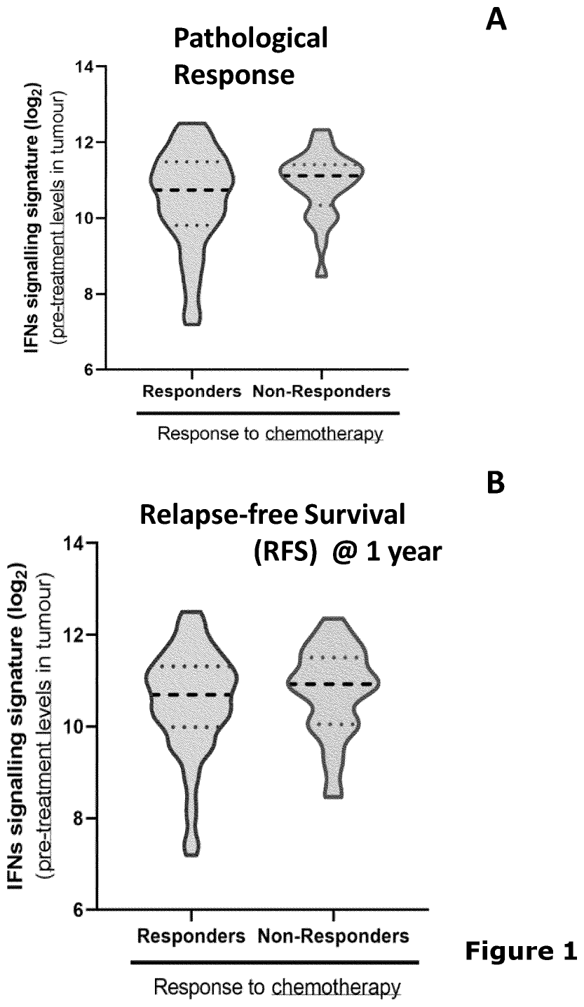

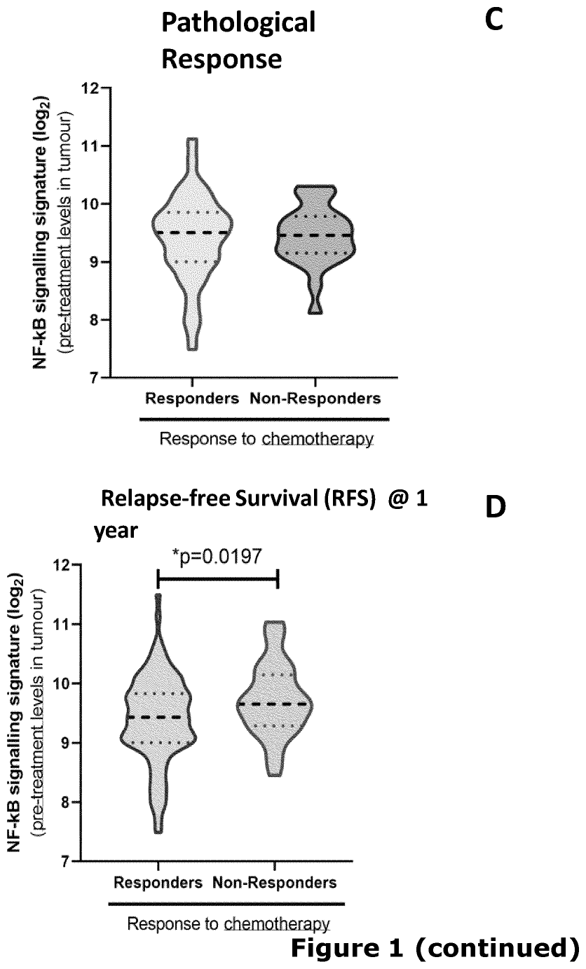

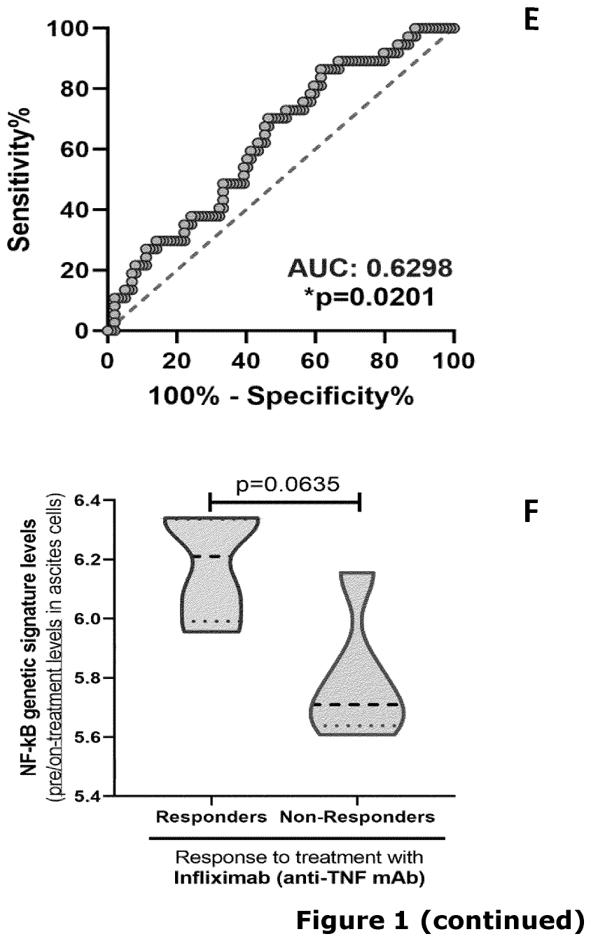

The distinctive predictive impact on OV-tumours associated with NFkB and IFNs signalling (genetic) signatures was investigated in OV patients. The ability to predict OV-tumours' short-term (i.e., pathological response or pR) and medium-term (i.e., ≥1 year long, relapse-free survival or RFS) responsiveness to standard-of-care (SOC) anti-OV chemotherapy was determined (e.g., chemotherapies based on platinum/taxane-agents, gemcitabine and topotecan). Pre-treatment tumour associated IFNs signalling signature levels failed to differentiate subsequent chemotherapy responders from non-responders in terms of both pR (FIG. 1A) or RFS (FIG. 1B) in OV patients. Herein, NFkB signalling signature phenocopied this “failure” on the level of pR (FIG. 1C) but on the level of RFS, NFkB signalling signature was indeed significantly upregulated in chemotherapy non-responsive OV-patients (FIG. 1D). Accordingly, pre-treatment tumour associated NFkB signalling signature levels were significantly predictive of non-responsiveness to SOC chemotherapy regimens (FIG. 1E). Remarkably, in line with this, OV patients responding favourably to anti-cytokine immunotherapy aiming to disrupt this pro-tumorigenic NFkB signalling (via anti-TNF antibody: infliximab), exhibited heightened expression of the NFkB signalling signature in pre-treatment OV-tumour associated ascites (FIG. 1F). Together these results further underscore the significant pro-tumorigenic impact of cytokine-driven NFkB signalling in OV and its ability to act as negative prognostic/predictive biomarker in OV patients. However, these results also outline that genetic signatures, while capable of estimating NFkB signalling, are not powerful enough to estimate the impact of IFNs signalling in predictive biomarker settings. Moreover, these genetic signature-based analyses are tumour-based and thus may or may not be applicable to patient serum, the extrapolation toward which is entirely impossible based on tumour data. This situation henceforth requires a dedicated serum-screening assay like the present sFIS assay.

The present invention illustrates that a “core” ISB-based phenotype in OV patients is best captured by a human myeloid cells-based functional biomarker screening “platform” that can simultaneously capture the induction of NFkB as well as IFNs responses in myeloid cells exposed to patient's serum-associated peripheral inflammatory factors thereby giving the best integrated serum-functional immunological status (sFIS) for OV patients.

THP1 binary-reporter cells such as used in example 1 were suitable for the present sFIS assay. These selection/testing criteria and such assessment workflow can be eventually applied to test any such reporter system, whether procured commercially or generated in-house, for suitability in running the present sFIS assay. As long as such criteria are met, any reporter system can be utilized for running this sFIS assay. The present invention shows such as disclosed in the examples can faithfully capture the functional immunological disbalance in the serum immunobiology of OV patients and thereby more directly emphasize the inflammatory (NFkB response>IFNs response) vs. immunogenic (IFNs response≥NFkB response) ISB-based phenotype characteristics.

The present invention shows that ISB-based phenotype estimation has better utility in predicting medium-to-long term survival of OV-patients (i.e. progression-free survival or PFS/overall survival or OS) rather than short-term responses (pR). This provides an advantage since the current serum biomarker analyses state-of-the-art already has a well-established biomarker for predicting short-term pR (i.e. serum-associated OV antigen, CA125). The present invention provides a reliable biomarker modality for predicting long-term patient survival on the basis of serum profiling. The present invention also show that negative prognostic/predictive biomarkers tend to have a more consistent biomarker performance in OV-patients irrespective of therapy subgroups whereas positive prognostic/predictive biomarkers may show therapy-dependent variability and importantly, both these complicated trends can be simultaneously as well as sufficiently captured by the present sFIS assay, much better than CA125 analyses (for PFS/OS estimation), thereby further outlining the versatility of the present assay.

### EXAMPLE 1: FOUNDATIONAL PRINCIPLE AND OPTIMIZATION BEHIND SFIS ASSAY

A 96-well plates-based throughput sFIS assay is based on human myeloid (monocytic) THP1 cell line stably expressing two inducible reporter constructs encoding for: (1) a luciferase gene (coding for a secreted form of luciferase enzyme) under the direct control of a dedicated promoter sequence linked to IFN-stimulated response element (ISRE) sequences; and (2) a secreted embryonic alkaline phosphatase (SEAP) reporter gene (coding for the widely utilized SEAP enzyme, a truncated form of GPI-anchored placental alkaline phosphatase) under the control of a dedicated promoter linked to c-REL binding site as well as NFKB consensus transcriptional response element. Of note, these inducible reporter constructs are interchangeable or even replaceable for other reporter outputs (e.g. fluorescence reporters, various luciferases and other colorimetric substrate-generating enzymes), as long as two distinct (inducible) reporter systems are separately linked to NFkB or IFN signalling pathway thereby allowing their distinct assessment, when both reporter systems are in the same cell line. Also, such constructs do not need to be expressed by the same THP1 cell line and can in fact be expressed by separate THP1 sub-cell lines. These THP1 reporter cells are supposed to sense functional cytokines, inflammatory factors, pattern-recognition receptor (PRR) agonists or IFN cytokines, thereby engaging the NFkB and/or IFN responses, resulting in secretion of SEAP/luciferase into the extracellular medium (in parallel to transcription of canonical NTGs or ISGs programs) thereby faithfully reporting commencement of NFkB-driven and/or ISRE-based transcriptional programs. By providing the proper substrate solutions for these enzymes, simultaneous detection of these two functional immunological readouts can be achieved per sample (colorimetric for SEAP and bioluminescence for luciferase enzyme; or other desired readouts as feasible or suitable so long as they are completely distinct i.e. fluorescence vs. bioluminescence vs. colorimetric).

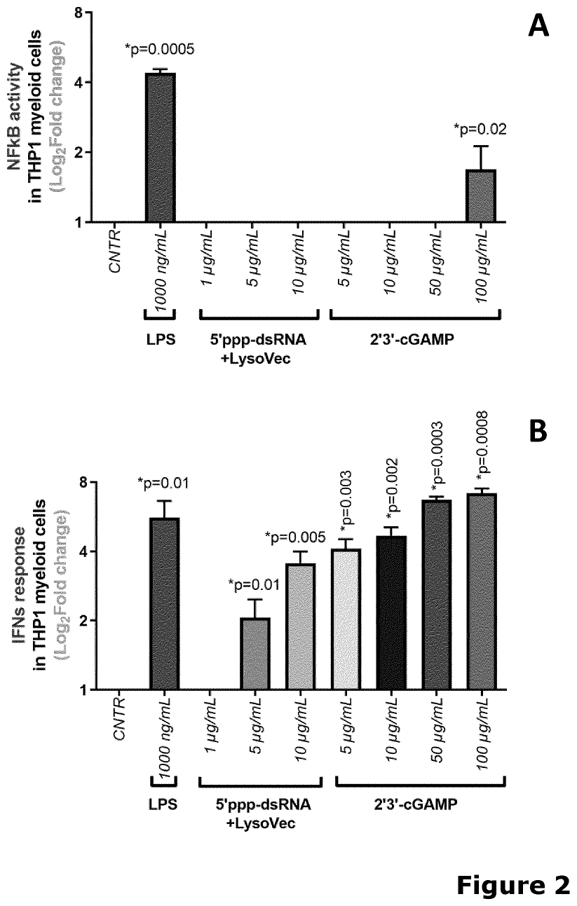

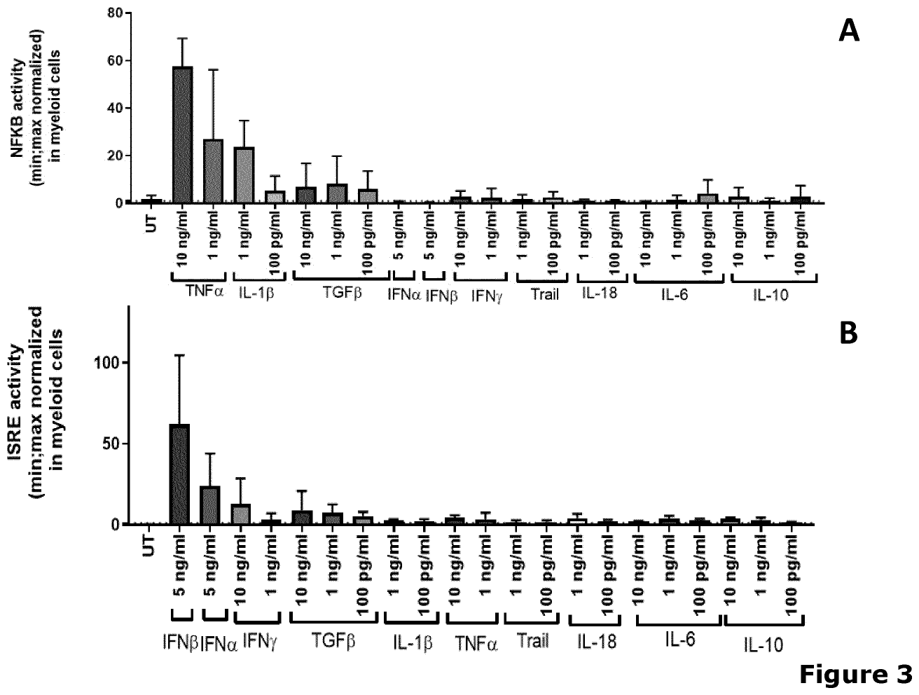

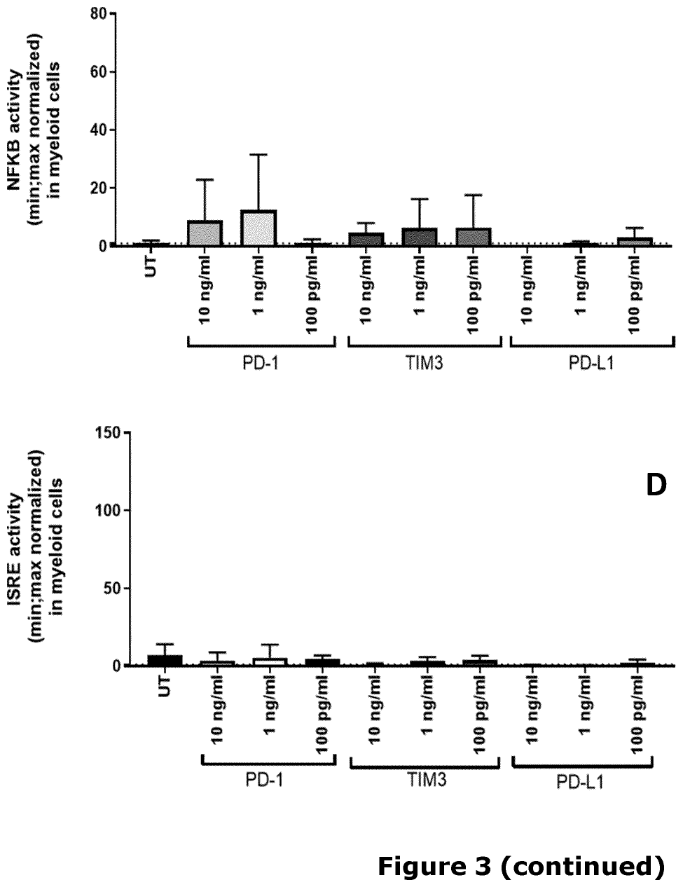

Accordingly, exposing these THP1 binary-reporter cells in a control experiment to standard PRR agonists like lipopolysaccharide (LPS: a TLR4 agonist), 5′ppp-dsRNA bound to a transfection reagent LysoVec (a RIG-I agonist) and 2′3′-cGAMP (a STING agonist) differentially (and sometimes distinctively) stimulated the NFkB (FIG. 2A) and IFNs (FIG. 2B) response reporter systems such that while LPS was highly proficient at activating both signalling modalities (FIG. 2A-B) yet RIG-I or STING agonists were (expectedly) better at inducing IFNs response than NFkB response (FIG. 2A-B). Similarly, treatment of these THP1 binary-reporter cells with canonical cytokine-based inducers of NFkB signalling pathway (TNF and IL1β) (FIG. 3A) or IFNs response pathways (IFNβ, IFNα and IFNγ) (FIG. 3B) resulted in either NFkB response or IFNs response reporter activity, respectively, with little redundancy amongst them.

In general, TRAIL, IL18, IL6 and IL10 did not sufficiently activate either of the transcriptional programs; although interestingly TGFβ a well-known pleiotropic cytokine induced threshold levels of both NFkB and IFNs responses (FIG. 3A-B). One emerging category of serum-associated biomarkers in immune-oncology context, are soluble immune-checkpoints (e.g. TIM3 or PD1/PD-L1). These reporter cells were exposed to recombinant PD1, TIM3 or PD-L1 proteins and observed that whereas they were in general incapable of substantially inducing either of the signalling programs yet recombinant TIM3 (and to a very small extent PD1) induced threshold (but non-significant) levels of NFkB response (FIG. 3C-D). Overall, this shows the wide-ranging sensitivity as well as robustness of this THP1 binary-reporter system in differentiating between general inflammatory stimuli (LPS/STING agonists, TGFβ) and specific immunological stimuli based on cytokine-cytokine receptor agonism (ILs vs. IFNs) on both qualitative as well as quantitative levels. Thus, these THP1 binary-reporter cells were suitable for the present sFIS assay. These selection/testing criteria and such assessment workflow can be eventually applied to test any such reporter system, whether procured commercially or generated in-house, for suitability in running the present sFIS assay. As long as such criteria are met, any reporter system can be utilized for running this sFIS assay.

### EXAMPLE 2: EXPOSURE OF SFIS ASSAY TO CANCER PATIENTS' SERUM

The biomarker efficiency of serum-induced NFkB or IFNs response through a “prospective retrospective” biomarker analyses strategy [Henry & Hayes (2012) Mol. Oncol. 6, 140-146; Simon et al. (2009) J. Natl. Cancer Inst. 101, 1446-1452] was tested on 98 archived serum specimens derived from randomly selected 32 OV-patients throughout their disease course at UZ Leuven, Belgium (hereafter referred to as the “UZL-CSI OV cohort”). For validating ISB-based phenotype-defining biomarker trends via sFIS assay, the UZL-CSI cohort was highly suitable since it provided the necessary immunological “dynamism” in terms of overall serum context. This is because it was composed of a diversity of pre-treatment (at primary/recurrence diagnosis or untreated recurrence stages) as well as on/post-treatment (with standard-of-care chemotherapies or specific clinical trials-related targeted or immune-therapies) serum specimens.

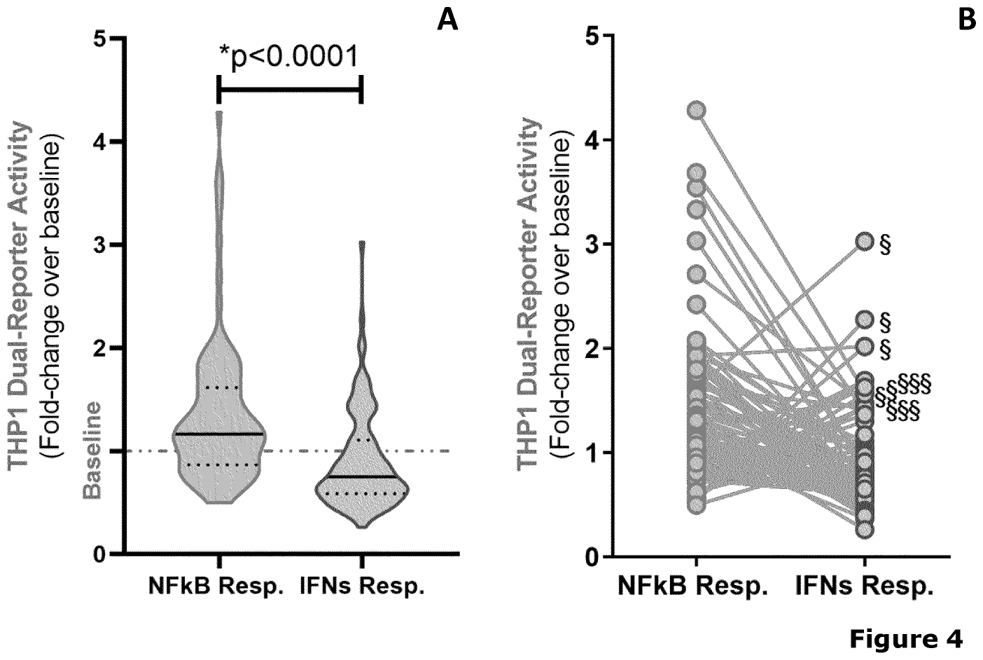

Remarkably, serum derived from these OV-patients indeed induced significantly higher NFkB responses in the THP1 binary-reporter cells as compared to IFNs responses (FIG. 4A). In fact, the median IFN responses induced by the OV-patient sera were below the present assay's baseline (i.e. normal human serum pooled from several healthy donors) thereby suggesting a tendency of most serum-associated factors to downregulate immunogenic IFNs signalling induction. In fact, there were only 11 serum specimens (out of 98) that induced a higher fold-change of IFN responses than NFkB responses (relative to baseline) in the THP1 binary-reporter cells (FIG. 4B). These results clearly outline that the present binary sFIS assay can faithfully capture the functional immunological disbalance in the serum immunobiology of OV patients and thereby more directly emphasize the inflammatory (NFkB response>IFNs response) vs. immunogenic (IFNs response NFkB response) ISB-based phenotype characteristics.

### EXAMPLE 3: THE SFIS ASSAY-BASED PREDICTION OF CANCER PATIENT SURVIVAL

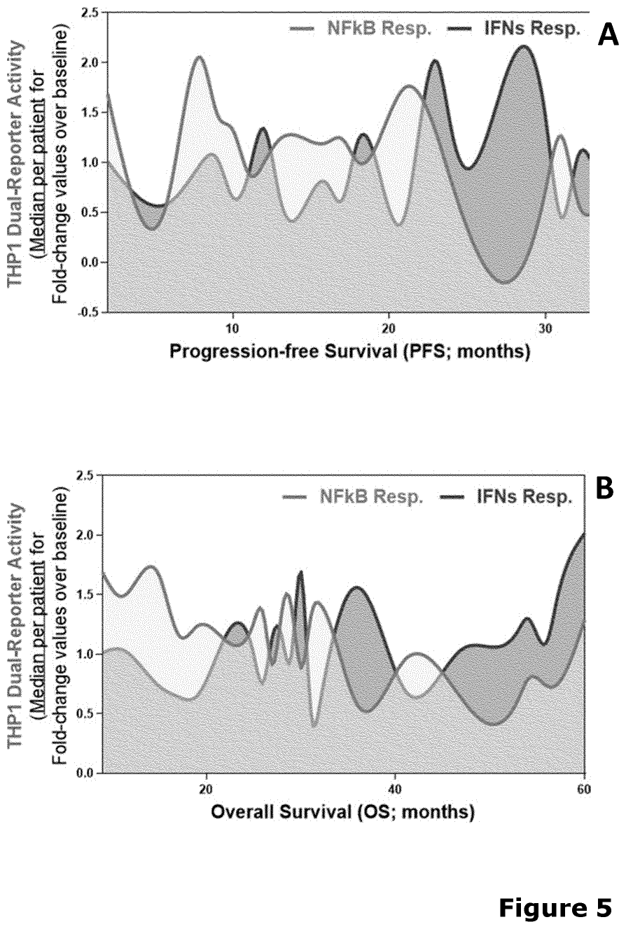

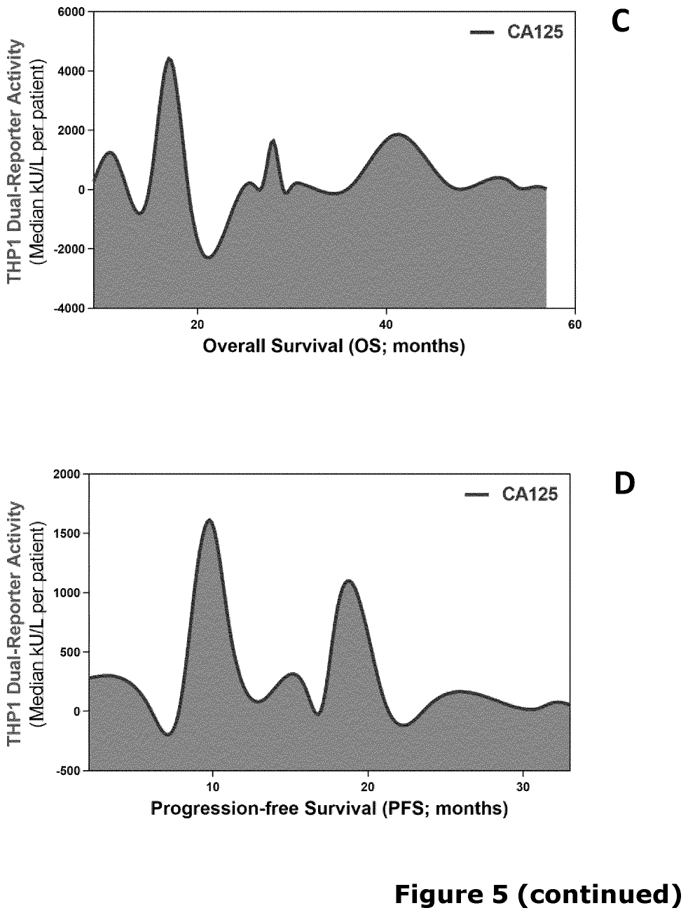

An immune signalling-amplitude analysis for NFkB/IFN responses generated via the present sFIS assay showed that OV-patients that have prolonged survival (PFS or OS; but especially OS) (FIG. 5A-B) and reduced/stabilized CA125 (an approved serum biomarker used as standard in ovarian cancer patients) serum levels (indicative of reduced or controlled OV-tumour burden) (FIG. 5C-D) had a more pro-immunogenic orientation between IFNs and NFkB responses (i.e., IFN Resp.>NFkB Resp.) whereas patients with a clear disbalanced orientation between these two inflammatory modules (NFkB Resp.≥IFN Resp.), had much shorter survival (especially OS) and mostly increased CA125 serum levels (FIG. 5A-D). Overall, the present sFIS assay is well attuned to robustly capturing the functional ISB-based phenotype thereby clearly outlining the ability of the present sFIS assay to capture both anticipated and possibly unanticipated serum-associated functional immunobiology.

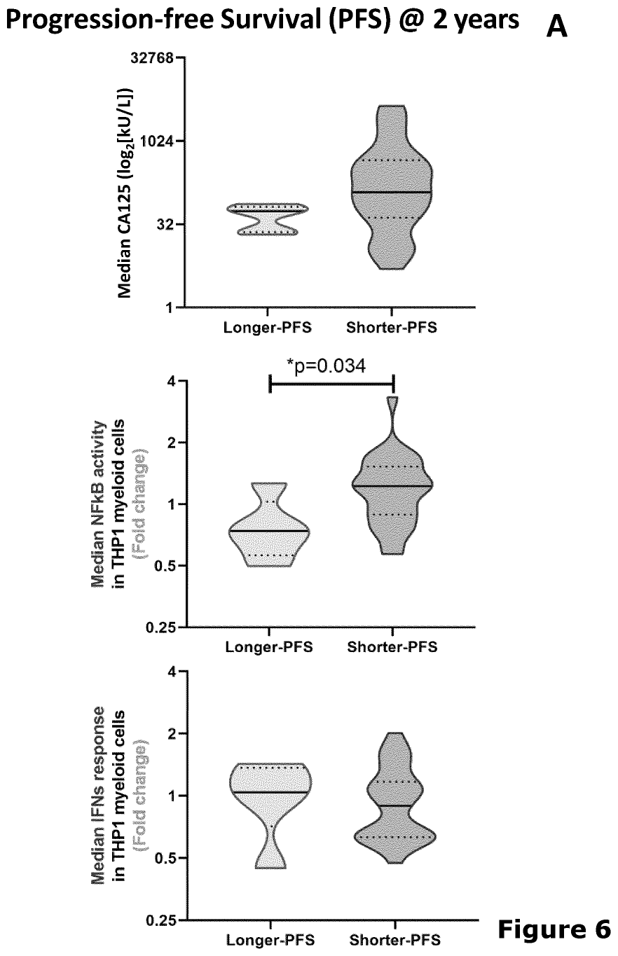

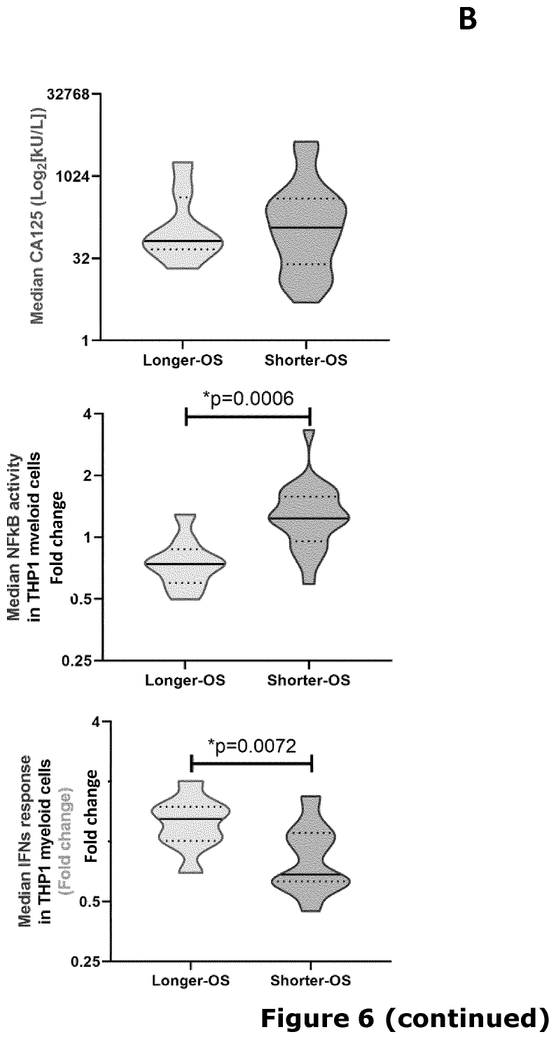

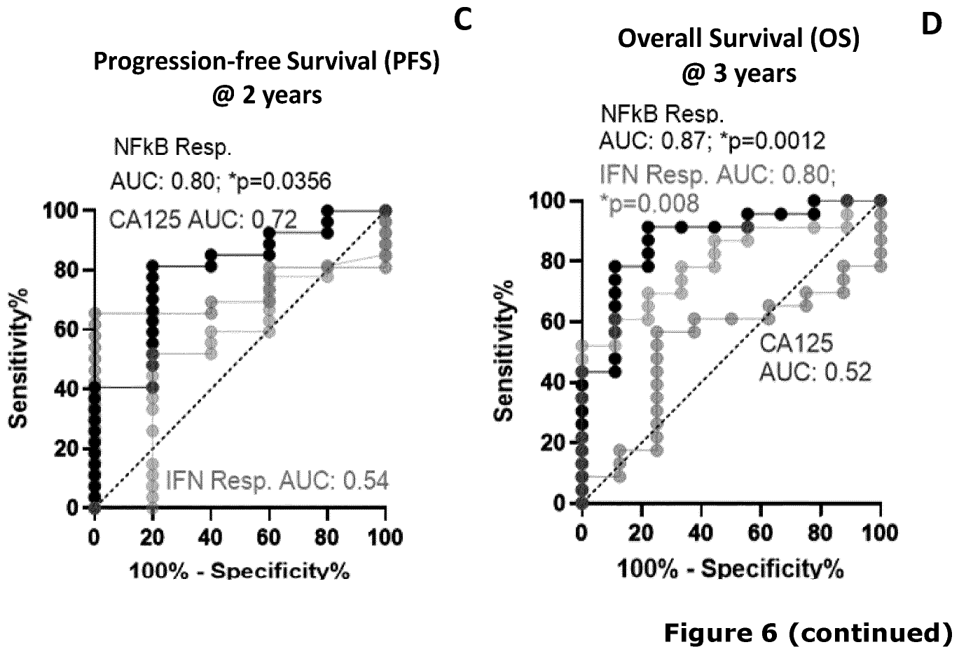

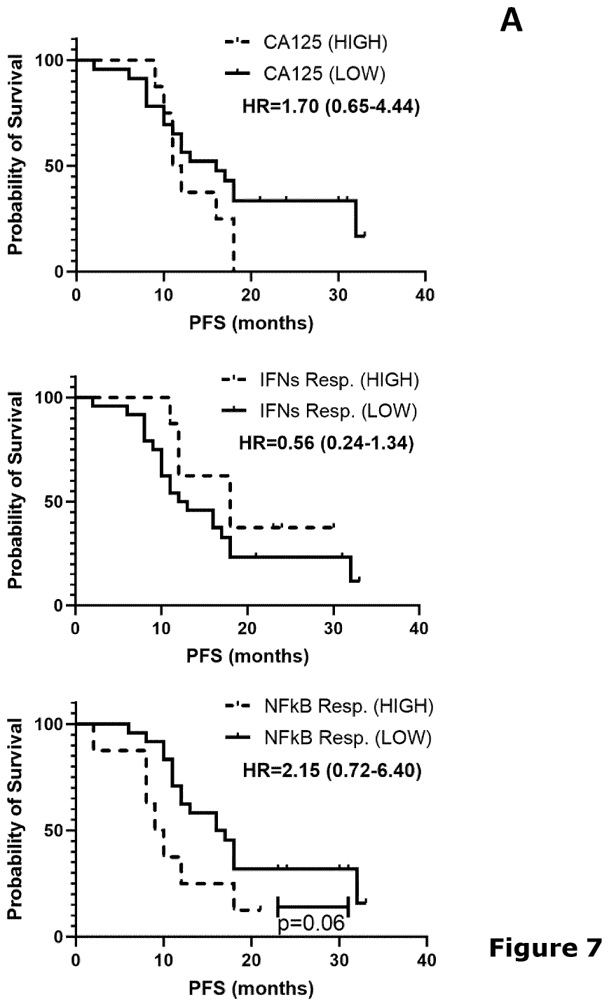

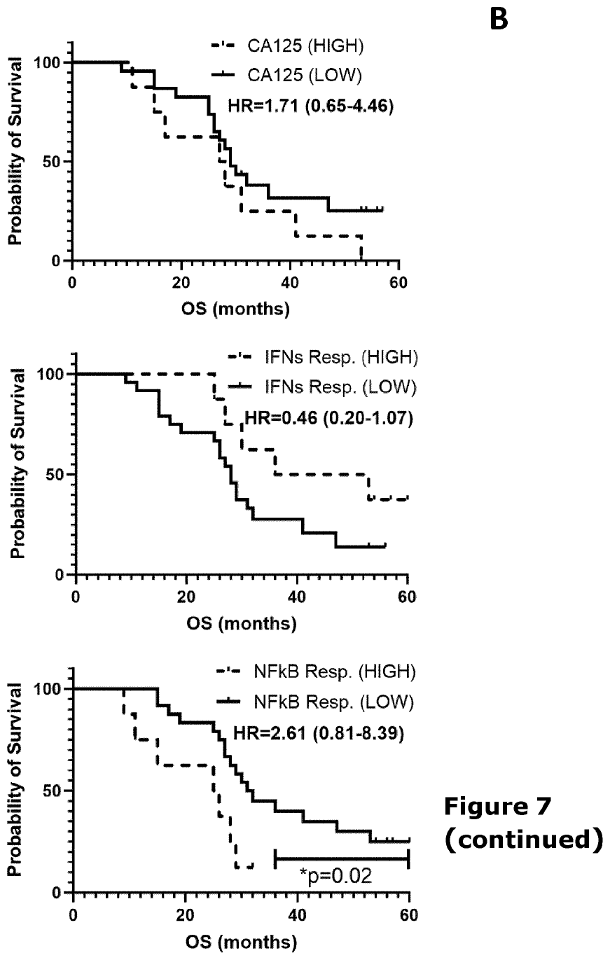

The above observations evidently outlined the superiority of the present sFIS assay in reliably mapping ISB-based phenotype, especially on the level of serum-associated functional immunobiology and immune-signalling amplitudes. Next, the biomarker efficacy of NFkB and IFN responses derived from the present sFIS assay were tested. The OV-patients with shorter PFS/OS had serum that induced significantly higher NFkB responses in the present sFIS assay whereas OV-patients with longer-PFS/OS had serum that induced higher IFN responses (although statistical significance was only reached for longer OS) (FIG. 6A-B). Herein, heightened serum CA125 levels were not conclusively indicative of differential PFS/OS (FIG. 6A-B). Accordingly, sFIS-derived NFkB response was a significant predictive biomarker for both medium-term (PFS) and long-term (OS) OV-patient survival whereas IFN responses were only significantly predictive for long-term OS (FIG. 6C-D); while CA125 could only partially predict differential PFS, but not OS (FIG. 6C-D). These trends were also supported by Kaplan-Meier (KM) survival analysis of OV-patients' PFS (FIG. 7A) or OS (FIG. 7B), such that very high serum-induced NFkB responses and serum CA125 levels acted as negative prognostic factors whereas very high serum-induced IFN responses acted as positive prognostic factor. Overall, this clearly establishes the prognostic biomarker efficacy of the present sFIS assay (which simultaneously integrates a negative as well as positive prognostic factor within a single platform) in predicting medium/long-term survival of OV-patients, sometimes even better than the standardly used biomarker in ovarian cancer i.e., serum CA125.

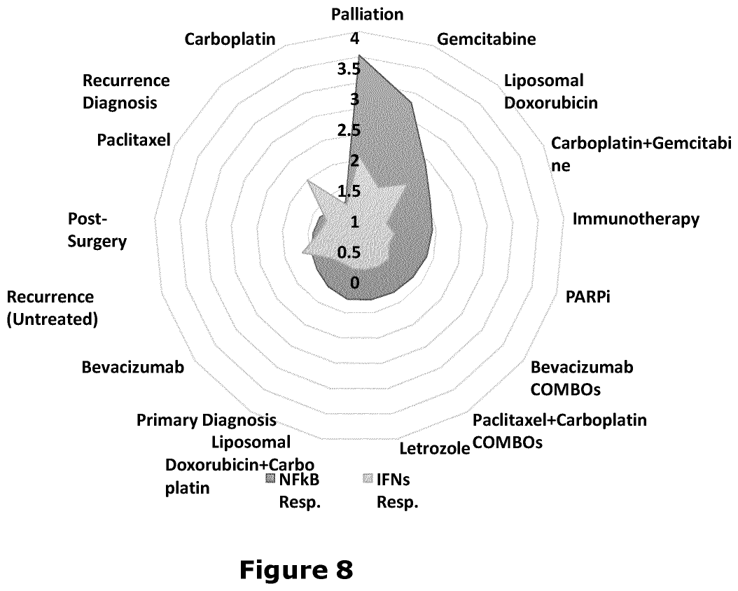

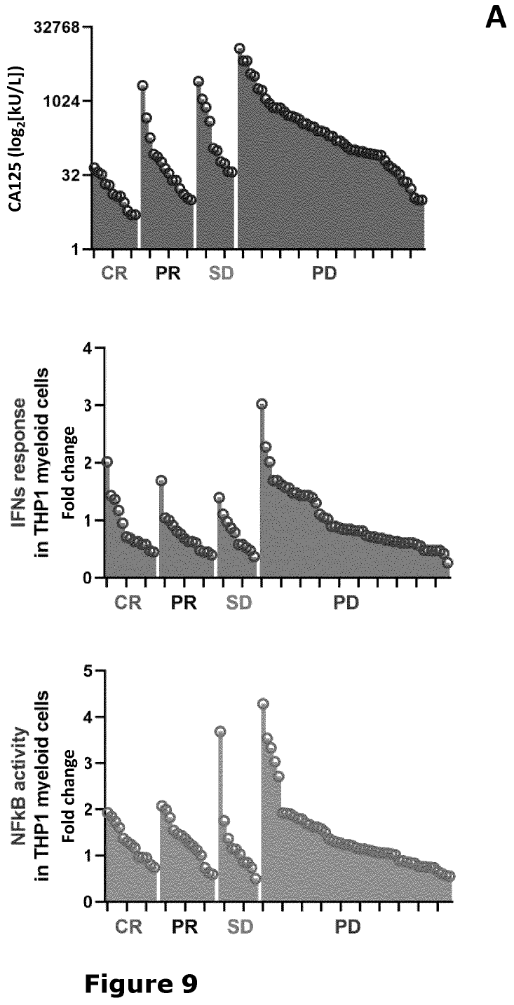

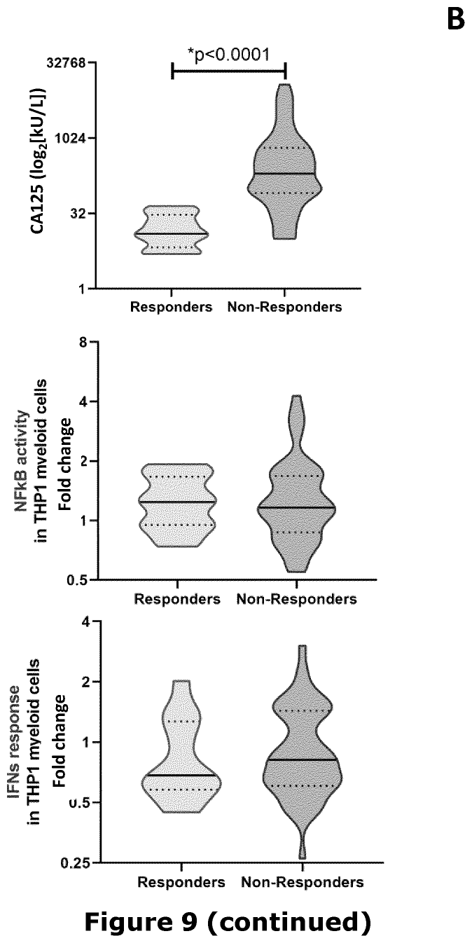

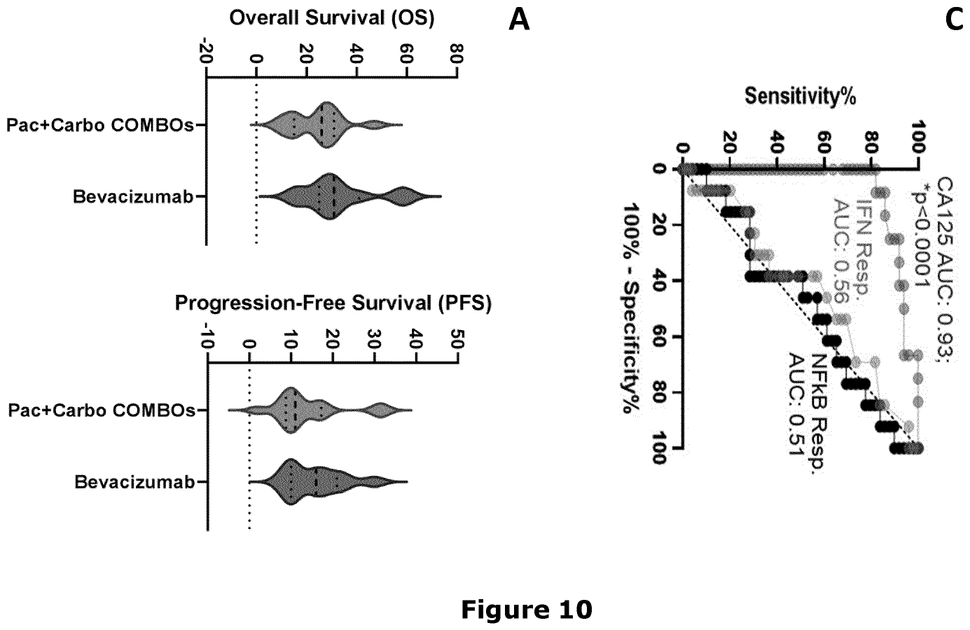

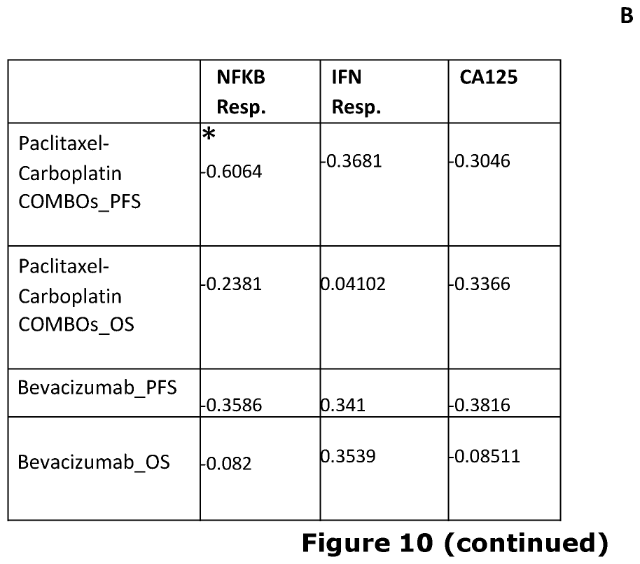

As mentioned previously the UZL-CSI cohort was composed of serum specimens derived from heterogeneous clinical settings for OV-patients including those derived from patients on- or post-anti-OV therapies. To this end, it was investigated how anticancer therapy was affecting NFkB or IFN responses generated by the sFIS assay. The present sFIS assay showed more differential inflammatory trends between different treatment or non-treatment ovarian cancer patient subgroups such that, most untreated subgroups had more IFN responses than NFkB responses (especially, at recurrence diagnosis and pre-treatment recurrence stages) (FIG. 8). Contrary to this, a number of anticancer therapies had a tendency to strongly increase NFkB response-inducing peripheral inflammatory factors (without necessarily potentiating IFN responses) in OV-patients serum (especially, gemcitabine, liposomal doxorubicin, carboplatin+gemcitabine, immunotherapy, PARPi and bevacizumab COMBOs) (FIG. 8), in line with the well-known tendency of anticancer therapies to potentiate systemic inflammation in cancer patients that might be pro-tumorigenic as reported previously [Ritter et al. (2019) J. Exp. Med. 216, 1234-1243; Nakamura & Smyth (2020) Cell Mol Immunol 17, 1-12; Hwang et al. (2011) BMC Cancer. 11, 489]. In order to allow a more targeted estimation of biomarker efficacy of the present sFIS assay relative to CA125, general pR trends were looked at in more detail (i.e. complete response or CR, partial response or PR, stable disease or SD or progressive disease or PD). Herein, in line with the standard observations, serum CA125 level was largely reduced in OV-patients showing CR, whereas it was strongly upregulated in other pR subgroups, especially PD (FIG. 9A). Accordingly, non-responder OV-patients (i.e. those experiencing PD at the time of serum specimen collection) had significantly higher CA125 serum levels than responder OV-patients (i.e. those experiencing CR at the time of serum specimen collection) (FIG. 9B). Similar differentiating trends for pR subgroups were not clearly visible for the present sFIS assay (NFkB/IFN response) readouts (FIG. 9A-B) thereby making serum CA125 levels the most superior predictor of pR in OV-patients as compared to the present sFIS readouts (FIG. 9C). From earlier predictive biomarker analyses for OV-patients subsequently treated with chemotherapy, it was also observed that tumour transcriptome-derived NFkB signalling signature was a better predictor of patient RFS rather than short-term pR. It was investigated if the same situation applied to the present sFIS assay readouts. To systematically unable this analyses, it initially broadly differentiated the OV-patients on the basis of pR i.e. CR+PR versus SD+PD, and observed that the highest amounts of favourable CR+PR responses where achieved in paclitaxel+carboplatin COMBOs subgroup whereas the highest amounts of unfavourable SD+PD responses where achieved in bevacizumab subgroup. However, in line with known inability of such anti-OV therapies in sufficiently improving long-term OV tumour progression despite providing short-term relief on the level of pathological tumour burden responses; the OS or PFS of OV-patients was not clearly increased in the paclitaxel+carboplatin COMBOs, as compared to bevacizumab (FIG. 10A). Nevertheless, a correlation analyses with PFS/OS, in these two therapeutic subgroups, showed that sFIS-derived NFkB responses (in general) negatively correlated with PFS/OS irrespective of therapy subgroup (similar to CA125) (FIG. 10B). In contrast, sFIS-derived IFN responses showed contrasting correlations depending on the therapy subgroup (FIG. 10B). Firstly, these trends confirm that the present sFIS assay-based ISB-based phenotype estimation has better utility in predicting medium-to-long term survival of OV-patients (i.e. PFS/OS) rather than short-term responses (pR). Indeed, the current serum biomarker analyses state-of-the-art already has a well-established biomarker for predicting short-term pR (i.e. serum CA125) Secondly, these trends also show that negative prognostic/predictive biomarkers tend to have a more consistent biomarker performance in OV-patients irrespective of therapy subgroups whereas positive prognostic/predictive biomarkers may show therapy-dependent variability and importantly, both these complicated trends can be simultaneously as well as sufficiently captured by the present sFIS assay, just as well as CA125 analyses, thereby further outlining the versatility of the present assay.

### EXAMPLE 3: EXPERIMENTAL DETAILS

Day 1.

Step 1. Scrape the semi-adherent cultures of the NFkB (SEAP reporter enzyme) and IFNs (LUCIA reporter enzyme) response (binary) genetic reporter THP1 human myeloid cells (in this case commercially procured from Invivogen; but similary cells are available from several other manufacturer's or can also be generated in-house) off the regular/routine cell culture flask(s) (while still in their selection media) with a standard cell scraper (Sarstedt).

Selection medium is THP1 cell culture medium with selection antibiotics: RPMI 1640, 2 mM L-glutamine, 25 mM HEPES, 10% heat-inactivated fetal bovine serum, 100 μg/ml Normocin, Pen-Strep (100 μg/ml), 10 μg/ml Blasticidin and 100 μg/ml of Zeocin.

Step 2. Count the amount of live THP1 cells through standard cell counting methodology.

Step 3. Plate 35000 THP1 cells in each well of a standard 96-well flat-bottom plate (in 100 μL cell culture medium*). Plate enough wells, considering the number of patient serum samples to be tested as well as the following mandatory sFIS assay controls:
2× wells of untreated THP1 cells in their normal cell culture medium* (background control);
2× wells of bacterial lipopolysaccharide (LPS)-treated THP1 cells (positive control);
2× wells of normal human serum pooled from healthy donors (baseline control) (Sigma-Aldrich).

The THP1 cells for the sFIS assay itself should be plated in the normal cell culture medium without their specific selection antibiotics.*

Day 3.

Step 4. Add 100 μL of (freshly defrosted) patient serum per well (in the 96-well plate from Day 1) on top of the THP1 cells thereby making the total volume in each well, 200 μL (i.e., 100 μL patient serum+100 μL THP1 normal culture medium). Composition of the normal THP1 cell culture medium: RPMI 1640 media, 2 mM L-glutamine, 25 mM HEPES, 10% heat-inactivated fetal bovine serum (FBS), 100 μg/ml Normocin and Pen-Strep (100 μg/ml).

Step 5. To setup the sFIS assay controls add, to the corresponding wells (as plated on Day 1), 100 μl of the above THP1 normal culture medium (background control), THP1 normal culture medium containing 1000 μg/mL of LPS (positive control) or 100 μl of normal human serum (baseline control) (see Step 3 above).
All THP1 cells-containing wells should now contain 200 μL of equal/total volume.

Day 4.

Step 6. Take the 200 μL of media/serum mixtures off the respective wells from the corresponding 96-well plates after 24 h of incubation as initiated on Day 3. In parallel, thaw the Quanti-Blue and Quanti-Luc/Quanti-Luc GOLD reagents (stored at −20° C.) and let them normalize to room-temperature. Herein, Quanti-Blue (Invivogen) is a substrate for the SEAP reporter enzyme while Quanti-Luc or Quanti-Luc GOLD (Invivogen) are the substrates for the LUCIA reporter enzyme.

Step 7. Pipette 100 μL of this media/serum (into a new standard cell culture-grade clear bottom 96-well plate) and, add 100 μL of Quanti-Blue reagent (the substrate for the SEAP enzyme, reporting for activation of the NFkB activity; Invivogen), and incubate at 37° C. in standard cell culture incubator until the LPS positive control wells become medium-dark blue due to production of a colorimetric byproduct (takes approximately 4-6 h).

Step 7A. Read the SEAP activity via absorbance capture (655 nm wavelength) with a standard 96-well plate reader with default (bottom-reading mode) settings for colorimetric/absorbance reading (e.g. FlexStation, Molecular Devices).

Step 8. Pipette the other 100 μL of the media/serum (into a separate, white, opaque-bottom 96-well plate) and add 50 μL of Quanti-Luc or Quanti-Luc GOLD reagents (the substrates for the LUCIA enzyme, reporting for activation of the ISRE/IRF activity; Invivogen).

Step 8A. Read the LUCIA luciferase activity via bioluminescence (500 ms of integrated signal) immediately (very important if using Quanti-Luc rather than Quanti-Luc GOLD!) after adding the Quanti-Luc/Quanti-Luc GOLD reagents (with the above plate reader; default settings for bioluminescence detection and top-reading mode) and repeat this 2 times with a 5 min interval and select the readings with most stable values as judged on the basis of background/baseline/positive controls.

The raw data are now ready for further analyses.

Further analysis.
As a quality control for this sFIS assay, LPS-induced SEAP/LUCIA luciferase activity values should always be upregulated 2 folds above the background control values per 96-well plate.
Raw values of both NFkB activity (SEAP-colorimetric readout) as well as the IFNs activity (luciferease-bioluminescence readout) are normalized by making a fold-change calculation to the baseline control i.e. SEAP or LUCIA activity values are divided by baseline control values to derive the final sFIS assay readout values for these reporter systems.

The sFIS Assay Biomarker Cut-Offs:
The NFkB (SEAP) activity of ≥1.5 fold-change above the baseline control (or ≥75th percentile of overall data-points distribution in a patient screening dataset) acts as a negative prognostic/predictive biomarker for cancer patients.
The IFNs (luciferase) activity of ≥1.3 fold-change above the baseline control (or ≥75th percentile of overall data-points distribution in a patient screening dataset) acts as a positive prognostic/predictive biomarker for cancer patients.

### EXAMPLE 4: EFFICIENCY OF SFIS ASSAY RELATIVE TO IMMUNE PROGNOSTIC PARAMETERS

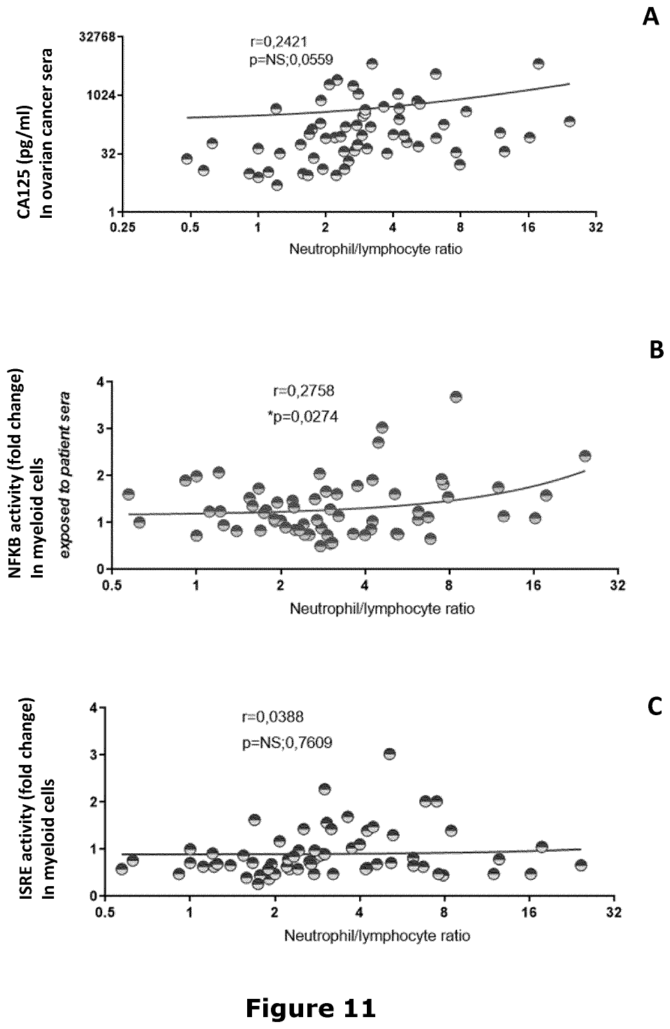

The above examples demonstrate the biomarker efficacy of the present sFIS assay with respect to well-established clinico-pathological estimators of patient survival or prognosis (pR, PFS or OS). Beyond these, there also exists an immunological parameter that predicts patient prognosis i.e., neutrophil-to-lymphocytes ratio (NLR), calculated by taking a ratio between absolute counts of neutrophils and lymphocytes estimated in the blood of the cancer patients. It is universally established that a very high NLR score predicts shorter survival in cancer patients thereby making it an important immunological negative prognostic parameter. To this end, it was evaluated how the present sFIS assay outputs align with NLR scores relative to the standard CA125 biomarker. In line with clinical experience and expectations, the serum CA125 antigen levels (indicative of high degree of tumour burden) in OV-patients positively correlated with higher NLR scores (FIG. 11A). In fact, NFkB responses induced by patient serum in the present sFIS assay also positively (and statistically significantly) correlated with high NLR scores (FIG. 11B) whereas serum induced IFN responses failed to show any correlation with NLR scores (FIG. 11C). Thus, even on a clinico-immunological level, sFIS-derived serum-induced NFkB responses, along with CA125 levels, were closely associated with ascending NLR scores thereby indicating worse OV-patient survival (FIG. 11A-B); whereas IFN responses didn't conform to this pattern in keeping with its positive prognostic behaviour. Overall, this clearly establishes the immuno-oncological biomarker efficacy of the present sFIS assay.

### EXAMPLE 5: TUMORAL IMMUNODYNAMICS OF NFKB/IFN RESPONSES PREDICT CANCER PATIENT'S SURVIVAL

Several studies have shown that origins of many (if not all) peripheral immuno-biomarkers in cancer patients can be traced to tumours. To appreciate if this also highlights a systemic inflammatory circuit on the level of NFkB/IFN responses, an existing scRNAseq map was probed that profiled tumour-infiltrating and (matched) blood-derived immune cells (procured from, renal cell or large cell neuroendocrine, carcinoma patients), with validated NFkB signalling or (type I/II) IFNs response-associated genetic signatures Within tumour-infiltrating immune cells, NFkB/IFN response signatures were mainly expressed by myeloid cells, which could also be traced to at least a small subset of peripheral myeloid cells, thereby implying a myeloid-level NFkB/IFN response circuit between the tumour and the periphery. To this end 12 TCGA-datasets were selected (spanning>5000 cancer patients) with diverse solid-tumours typically showing either immunotherapy-responsiveness (i.e., lung cancer, LUAD/LUSC; head & neck cancer, HNSC; bladder cancer, BLCA; renal cell cancer, KIRC; liver cancer, LIHC), or immunotherapy-resistance (ovarian cancer, OV; endometrial cancer, UCEC; sarcoma, SARC; breast cancer, BRCA; pancreatic cancer, PAAD; cervical cancer, CESC). Likewise, these immuno-biomarkers exhibited a highly variable cancer-type dependent prognostic impact on patient overall survival (OS). These observations exposed the considerable expressional (quantitative) and functional (effect on OS) heterogeneity or contradictions amongst major immuno-biomarkers. Next, it was investigated whether the more simplified ‘dynamics’ of the binary NFkB/IFN responses may better summarize a general inflammatory status than these heterogenous patterns of individual immuno-biomarkers. To address this, the median expression and median hazard ratios (HR) were derived for each of the highly expressed immuno-biomarkers across all 12 cancer-types and applied dimensionality reduction (PCA). This consolidated two distinct clusters i.e., a cluster of immuno-biomarkers with negative prognostic impact (i.e., high gene expression=OS↓), and those with positive (i.e., high gene expression=OS↑). Subsequently, the literature-driven annotation of the immuno-biomarker genes within these clusters for major NFkB target genes (NTGs) or IFNs stimulated genes (ISGs) showed that, negative prognostic immuno-biomarkers ‘enumerated’ a disbalance in these signalling pathways in favour of NFkB signalling (NTGs>ISGs), whereas positive prognostic immuno-biomarkers tended to have a relatively better balance (NTGs≈ISGs) between these signalling modalities. Thus, a disbalance in tumoural immuno-dynamic signalling that favours NFkB responses predicted shortened patient survival whereas more balanced signalling between IFN-NFkB responses predicted prolonged patient survival.

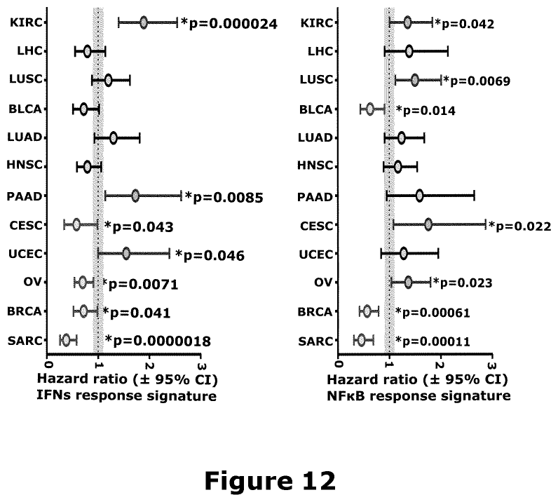

The prognostic impact of tumoral NFkB/IFN response signatures on patient OS was investigated in the above 12 cancer-types, to delineate which cancer-types might best capture the contrasting interplay between NFkB (negative prognostic) and IFN (positive prognostic) responses (FIG. 12). Herein, predominantly CESC or OV patients exhibited (statistically significant) contradictory prognostic impacts for NFkB and IFN response signatures (FIG. 12). Similar trends were also observed for LIHC and HNSC (FIG. 12).

### EXAMPLE 6 PERIPHERAL IMMUNODYNAMIC STATUS CAN GUIDE CHEMO-IMMUNOTHERAPY REGIME'S DESIGN

OV-patients exhibiting a bad prognostic, si-NFkB response^{HIGH}si-IFN response^{LOW/NULL} status, is consistent with OV's immuno-resistant nature. To this end, it was investigated whether blunting si-NFkB response while potentiating si-IFN response can be utilized as a guiding strategy to design precision combinatorial immunotherapy regime against OV. To address therapeutic inhibition of si-NFkB response, we pursued an in-silico drug-prediction relying on a computational algorithm exploiting biomedical literature-associated drug-gene relationships to predict drugs or drug-target's associations to the NFkB response-signature. Interestingly, this analyses recurrently enriched for anti-cytokine immuno-therapies or inflammatory therapeutic-targets, especially TNF inhibitory/blocking therapeutics like TNF-alpha inhibitors or infliximab (an anti-TNF antibody), to broadly target the NFkB response. This was concurrent with the observation that, amongst various cytokines that were screened, TNF was the most robust NFkB response-inducer.

The notion of applying anti-cytokine immunotherapies like anti-TNF antibodies is not new. In fact such anti-cytokine immunotherapies have shown recurrent success in preclinical studies whilst failing to reach similar success in clinical trials, across various cancers including OV. Thus, it has been proposed that the success of anti-cytokine immunotherapy might be contingent on biomarker-driven application, however precision biomarkers guiding its application are elusive. It was investigated whether si-NFkB response can better guide application of anti-TNF immunotherapy in OV-patients. Re-analyses of a small (only existing) human OV clinical trial, administering infliximab, wherein pre/on-treatment transcriptomic profiles for ovarian cancer-ascites were available, showed that: TNF levels in ovarian cancer-ascites couldn't sufficiently differentiate infliximab-responding vs. non-responding patients, whereas heightened NFkB response signature relatively better differentiated infliximab-responders from non-responders.

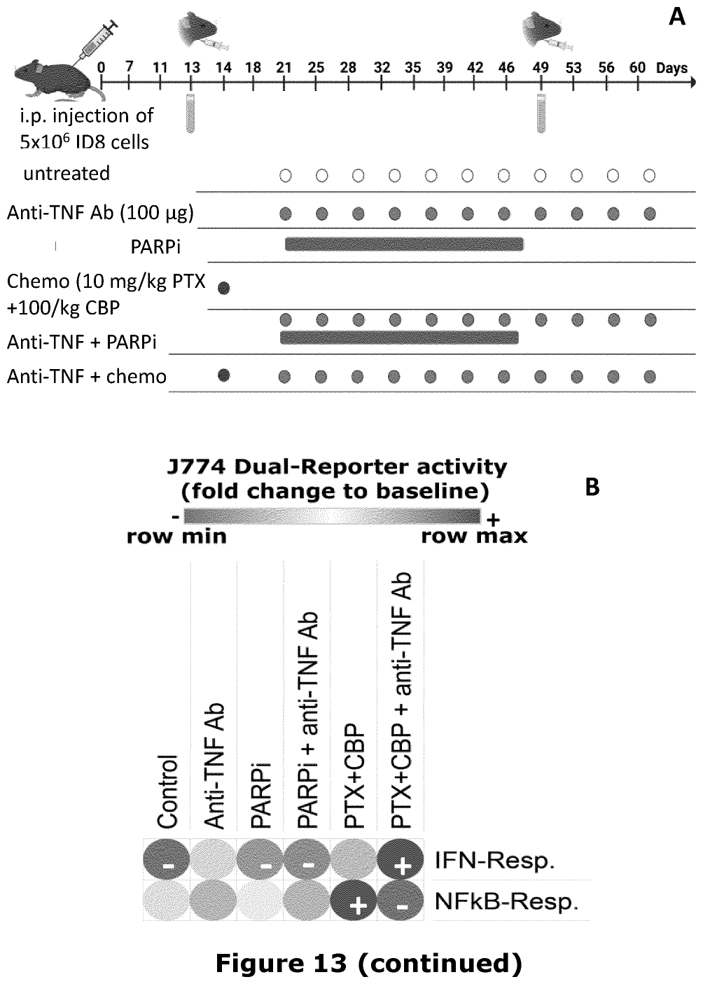

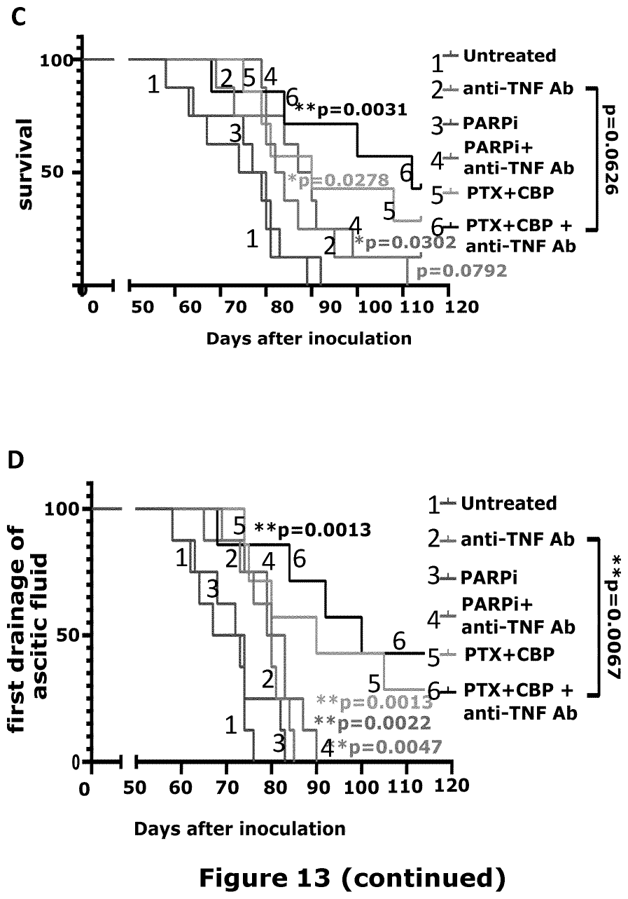

To validate these observations on the level of sFIS assay, the murine (preclinical) ID8 cells-based orthotopic model of metastatic OV was exploited, since these metastatic ID8-tumour bearing mice experience a latent (significant) spike in serum TNF-levels, post-tumour implantation. Herein, various (pre-)clinical studies have demonstrated anti-tumour efficacy as well as immunostimulatory or IFNs-inducing impact of (standard) anti-OV therapies like, combination of paclitaxel+carboplatin (PTX+CBP) chemotherapies, and/or PARP inhibitors (PARPi); yet it is not entirely clear which of these conventional therapies might best potentiate the anti-tumour efficacy of anti-TNF antibodies. It was further investigated whether sFIS assay may allow us to predict the anti-tumour efficacy of above therapeutics (alone or in combination with anti-TNF immunotherapy). ID8-tumour bearing mice were treated with PTX+CBP, PARPi, or anti-TNF immunotherapy alone, or in combinatorial regimens (i.e., PTX+CBP+anti-TNF antibody, or PARPi+anti-TNF antibody) (FIG. 13A). Initially, the murine serum was collected at baseline and after above therapeutic treatments (FIG. 13A) and screened the ability of these serum samples to induce si-NFkB/si-IFN responses using a murine version of our sFIS assay i.e., J774 murine myeloid cells stably expressing two inducible reporter constructs for NFkB response, or IFN response signalling. Interestingly, a ratio metric analyses of murine sFIS assay derived si-NFkB/si-IFN responses (post-to-pre treatment) showed that: anti-TNF immunotherapy exhibited threshold tendencies to reduce si-NFkB responses while marginally potentiating si-IFN responses (FIG. 13B). Remarkably, combining anti-TNF immunotherapy with PTX+CBP created a much better pro-immunogenic peripheral immunodynamic status (si-NFkB response^{LOW} si-IFN response^{HIGH}), than combining with PARPi (si-NFkB response^{MED} si-IFN response^{LOW}) (FIG. 13B). PARPi or PTX+CBP alone did not exhibit the same degree of pro-immunogenic peripheral potential as PTX+CBP+anti-TNF chemo-immunotherapy combination (FIG. 13B). Remarkably in terms of anti-tumour efficacy, in line with above sFIS assay-predictions, the ID8-tumour bearing mice treated with PTX+CBP+anti-TNF immunotherapy triple-combo (but not PARPi+anti-TNF immunotherapy) had the longest overall survival (FIG. 13C) as well as the longest survival relative to the first ascitic fluid's drainage (FIG. 13C); wherein, repeated ascitic fluid-drainage within our murine model was highly representative of standard clinical practice for OV-patients. PTX+CBP+anti-TNF chemo-immunotherapy had significantly higher anti-OV efficacy than untreated as well as anti-TNF immunotherapy treatment alone (FIG. 13C-D); thereby highlighting the superiority of PTX+CBP (over PARPi) in unleashing anti-TNF immunotherapy's potential. Overall, a ratio metric analyses established that a peripheral si-IFN response^{HIGH}si-NFkB response^{LOW} status positively correlated with prolonged survival patterns in preclinical OV-settings. In conclusion, the sFIS assay can be applied for designing anti-OV combo-therapy, especially those involving anti-TNF immunotherapy, with si-IFN response^{HIGH}si-NFkB response^{LOW} phenotype being the most immunogenic context.

1. An in vitro method for predicting tumour progression or response to anticancer therapy of a cancer patient, wherein the cancer is selected from the group consisting of ovarian cancer, cervical squamous cell carcinoma and endocervical adenocarcinoma (CESC), head and neck squamous cell carcinoma (HNSC) and liver hepatocellular carcinoma (LHC) the method comprising the steps of:
1) providing engineered mammalian immune cells comprising:
a NFkB signalling reporter construct comprising an NFkB signalling complex-responsive promoter sequence linked to multiple copies of c-REL binding site and multiple copies of the NFKB consensus transcriptional response element, which binds an NFkB transcription factor complex, fused to a gene sequence encoding a first reporter protein,
and
an IFN signalling reporter construct comprising an interferon-induced transcription factors-responsive promoter sequence linked to one or more copies of IFN-stimulated response element (ISRE) sequences, which binds IFN response transcription factors, fused to a gene sequence coding a second reporter protein,
2) contacting the engineered cells with a blood sample of a cancer patient, allowing induction of the NFkB and/or IFN signalling pathways of said engineered cells by the blood sample,
3) determining the expression and/or activity levels of the first and second reporter protein,
4) comparing the expression levels and/or activity of the first and second reporter protein determined in step 3) with the expression and/or activity levels of the first and second reporter protein in a reference sample of a healthy individual,
5) based on the comparison predicting tumour progression, or response to anticancer therapy of the cancer patient,
wherein an increase of activity or expression level of the first reporter protein for NFkB signalling compared to the reference value is a negative indicator of tumour progression, or response to anticancer therapy, and
wherein an increase of activity or expression level of the second reporter gene for IFN signalling compared to the reference value is a positive indicator of the tumour progression, or response to anticancer therapy.

2. The method according to claim 1, wherein the response to anticancer therapy predicts the life expectancy of the cancer patient after cancer therapy.

3. The method according to claim 1, wherein the prediction of tumour progression predicts the medium-to-long term survival of a cancer patient.

4. The method according to any one of claims 1 to 3, wherein the immune cells are human cells.

5. The method according to any one of claims 1 to 4, wherein the immune cells in step 1) are monocytes.

6. The method according to any one of claims 1 to 5, wherein a ≥1.5 fold-change of expression and/or activity of the first reporter protein for NFkB signalling, compared to the reference value, is a negative indicator of tumour progression or response to anticancer therapy.

7. The method according to any one of claims 1 to 5, wherein a ≥1.3 fold-change of expression level and/or activity of the second reporter protein for IFN signalling, compared to the reference value, is a positive indicator of the tumour progression or response to anticancer therapy.

8. The method according to any one of claims 1 to 7, where the reporter protein is selected from the group consisting a luciferase, a fluorescent or bioluminescent protein and an alkaline phosphatase.

9. The method according to any one of claims 1 to 8, wherein the expressed reporter gene is a secreted protein.

10. The method according to an one of claims 1 to 9, wherein the ovarian cancer is malignant ovarian cancer or high-grade serous ovarian carcinoma.

11. The method according to any one of claims 1 to 10, wherein the patient underwent an anticancer therapy selected from the group consisting of chemotherapy, radiotherapy, small-molecule inhibitors, targeted therapy, palliative therapy, alternative therapies and immunotherapy.

12. Use of an engineered mammalian cells comprising a NFkB reporter gene construct, and an IFN reporter gene construct,
a NFkB signalling reporter construct comprising an NFkB signalling complex-responsive promoter sequence linked to multiple copies of c-REL binding site and multiple copies of the NFKB consensus transcriptional response element binding an NFkB transcription factor complex, fused to a gene sequence encoding a first reporter protein,
and
an IFN signalling reporter construct comprising an interferon-induced transcription factors-responsive promoter sequence linked to one or more copies of IFN-stimulated response element (ISRE) sequences, which binds IFN response transcription factors, fused to a gene sequence coding a second reporter protein,
in predicting tumour progression or response to anticancer therapy of a cancer patient, wherein the cancer is selected from the group consisting of ovarian cancer, cervical squamous cell carcinoma and endocervical adenocarcinoma (CESC), head and neck squamous cell carcinoma (HNSC) and liver hepatocellular carcinoma (LHC).
